(19) United States
(12) Patent Application Publication
(10) Pub. No.: US 2003/0204190 A1
(43) Pub. Date: Oct. 30, 2003

(54) Bone expanding device and associated method of expanding a bone

(76) Inventor: Min Li, Indianapolis, IN (US)

Correspondence Address: BARNES & THORNBURG, 11 SOUTH MERIDIAN, INDIANAPOLIS, IN, 46204

(21) Appl. No.: 10/135,477

(22) Filed: Apr. 30, 2002

(51) Int. Cl. A61B 17/58

(52) U.S. Cl. 606/90

(57) ABSTRACT

A bone expanding device which includes a spontaneous biasing mechanism is disclosed.

### FIELD OF THE INVENTION

The present invention relates to a bone expanding device and an associated method of expanding a bone, and more particularly to a bone expanding device which is placed completely under the skin.

### BACKGROUND OF THE INVENTION

Early treatment for young children with craniofacial skeleton deformities remains a challenge. Existing treatments fall into two categories: osteotomy and distraction. Since the establishment of modern craniofacial surgery osteotomies have been applied to treat craniofacial skeleton deformities in infants and children, especially the deformities with short courses or threats to important organs such as multiple-suture synostoses and exorbitism. Although significant improvements have been realized by most children with severe craniofacial deformities treated with osteotomies, the long term results are difficult to predict due to limited understanding of craniofacial growth and development. Moreover, osteotomy procedures are traumatic, high risk, and time-consuming for young patients. Furthermore, negative psychological changes can develop as a result of some osteotomy procedures, some of which may require special psychological therapy.

An additional drawback to osteotomy procedures is that most congenital craniofacial skeletal deformities addressed with such procedures require repeated corrections before the active craniofacial growth slows. Repeating these procedures can cause increased scarring, which in turn increases the risk and difficulty of each subsequent procedure. Furthermore, multiple surgeries can result in long hospital stays, thereby resulting in tremendous financial pressure on both the patient's family and the insurance company.

With respect to conventional bone distraction, these procedures utilize a device which includes two pin-holders attached to a long screw. The device is secured to the bone so that a portion of the mechanism extends through the skin of the patient and is exposed to the exterior environment (i.e. external bone distraction). This exposed portion is then manipulated so as to exert a force on the bone to be distracted.

Bone distraction can be successful, however it does suffer from several drawbacks. For example, external bone distraction can result in visible scars, pin tract infection, and can have a negative psychological impact on the patient and the patient's family. Furthermore, external bone distraction devices are typically heavy mechanisms which makes it additionally uncomfortable for the patient. Moreover, multi-direction distraction is difficult to achieve with external distraction devices. In addition, the distraction of deeper structures, such as skull base, post-maxilla and orbits, are also difficult to achieve with external distraction devices.

What is needed therefore is a bone expanding device which addresses one or more of the above described drawbacks.

### SUMMARY OF THE DISCLOSURE

In one illustrative embodiment, there is provided an arrangement for expanding bone. The arrangement includes a bone distractor having (i) a first distractor body, (ii) a second distractor body mechanically coupled to the first distractor body so that the second distractor body is movable relative to the first distractor body between a first position and a second position, and (iii) a spontaneous biasing mechanism operatively coupled to the first distractor body and the second distractor body. The spontaneous biasing mechanism spontaneously urges the second distractor body from the second position to the first position when the second distractor body is located in the second position.

In another exemplary embodiment, there is provided a bone distractor. The bone distractor includes a first bone distractor body having a first opening which leads to a first chamber defined in the first bone distractor body. The bone distractor also includes a second bone distractor body having a second opening which leads to a second chamber defined in the second bone distractor body. The second bone distractor body is slidably positioned within the first chamber so that (i) the second opening is located within the first chamber and (ii) the second bone distractor body is positionable relative to the first bone distractor body between an extended position and a retracted position. The bone distractor further includes a spontaneous biasing mechanism operatively coupled to the first bone distractor body and the second bone distractor body. The spontaneous biasing mechanism spontaneously urges the second bone distractor body from the retracted position to the extended when the second bone distractor body is located in the retracted position.

In yet another exemplary embodiment, there is provided a method of expanding a bone of an animal. The method includes (a) inserting a bone distractor having (i) a first bone distractor body, (ii) a second bone distractor body mechanically coupled to the first bone distractor body so that the second bone distractor body is movable relative to the first bone distractor body between a first position and a second position, and (iii) a spontaneous biasing mechanism operatively coupled to the first bone distractor body and the second bone distractor body, wherein the spontaneous biasing mechanism spontaneously urges the second bone distractor body from the second position to the first position when the second bone distractor body is located in the second position, underneath tissue covering the bone of the animal, (b) securing a first attachment end and a second attachment end of the bone distractor to the bone such that the second bone distractor body is located in the second position relative to the first bone distractor body, and (c) covering the bone distractor with the skin so that the bone distractor is completely covered by the tissue.

In still another exemplary embodiment, there is provided a bone expanding device. The bone expanding device includes a first body having a first chamber defined therein. The bone expanding device also includes a second body having a second chamber defined therein. The second body is slidably positioned within the first chamber so that the second body is positionable relative to the first body between an extended position and a retracted position. The bone expanding device further includes a resilient body member located within the first chamber and the second chamber so that the resilient body member spontaneously urges the second body from the retracted position to the extended position when the second body is located in the retracted position.

The above and other objects, features, and advantages of the present invention will become apparent from the following description and the attached drawings.

### BRIEF DESCRIPTION OF THE DRAWINGS

FIG. 1 is a cross sectional view of an arrangement for expanding bone with the spontaneous biasing mechanism removed therefrom for clarity of description;

FIG. 2 is view similar to FIG. 1, but showing an example of a spontaneous biasing mechanism positioned within a first chamber and a second chamber of the arrangement;

FIG. 3 is a side elevational view of the arrangement of FIG. 1 located in an extended position;

FIG. 4 is a view similar to FIG. 3, but showing the arrangement in a retracted position;

FIG. 5 is an enlarged view of an arrangement for expanding bone secured to a bone;

FIG. 6 is an enlarged fragmentary view of an arrangement for expanding bone secured to a bone with brackets;

FIG. 7 is an enlarged fragmentary view of a pair of arrangements for expanding bone secured to a bone with brackets;

FIG. 8 is another enlarged fragmentary view of a pair of arrangements for expanding bone secured to a bone with brackets;

FIG. 9 is a perspective view of a human skull having a number of arrangements for expanding bone secured thereto;

FIG. 10A is an enlarged top elevational view of an attachment bracket which can be used to secure the arrangement of FIG. 1 to a bone;

FIG. 10B is an enlarged top elevational view of an attachment bracket configuration which can be used to secure the arrangement of FIG. 1 to a bone;

FIG. 10C is an enlarged top elevational view of another attachment bracket configuration which can be used to secure the arrangement of FIG. 1 to a bone;

FIG. 11 is a fragmentary cross sectional view of a bone covered by a tissue;

FIG. 12 is a view similar to FIG. 11, but showing an arrangement for expanding a bone being inserted underneath tissue covering the bone; and

FIG. 13 is a view similar to FIG. 12 but showing the arrangement for expanding the bone positioned completely underneath the tissue covering the bone.

### DETAILED DESCRIPTION OF ILLUSTRATIVE EMBODIMENTS

While the invention is susceptible to various modifications and alternative forms, specific exemplary embodiments thereof have been shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention as defined by the appended claims.

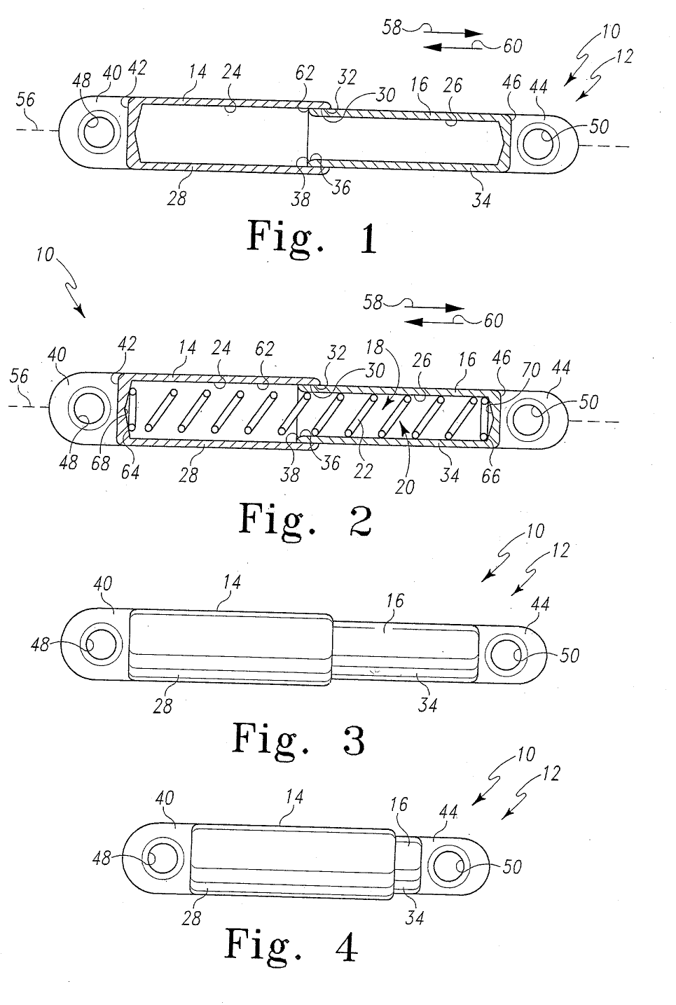

Referring now to FIGS. 1 and 2, there is shown an arrangement 10 for expanding a bone of an animal. For example, arrangement 10 can be utilized to lengthen a bone of an animal. Arrangement 10 includes a bone distractor 12. Bone distractor 12 includes a bone distractor body 14, a bone distractor body 16, and a spontaneous biasing mechanism 18. Bone distractor body 14 defines a substantially cylindrical sleeve 28 which defines a chamber 24. Sleeve 28 has an end 30 with an opening 32 defined therein. Opening 32 leads to chamber 24. Sleeve 28 also has an attachment end 42 with a bracket 40 extending therefrom. Bracket 40 has a screw hole 48 defined therethrough. In a similar manner, bone distractor body 16 defines a substantially cylindrical sleeve 34 which defines a chamber 26. Sleeve 34 has an end 36 with an opening 38 defined therein. Opening 38 leads to chamber 26. Sleeve 34 also has an attachment end 46 with a bracket 44 extending therefrom. Like bracket 40, bracket 44 also has a screw hole 50 defined therethrough.

Still referring to FIGS. 1 and 2, sleeve 34 is positioned relative to sleeve 28 such that a portion of sleeve 34 extends into chamber 24 of sleeve 28. In particular, sleeve 28 is positioned relative to sleeve 34 such that end 36 and opening 38 of sleeve 34 is located within chamber 24 so that chamber 24 and chamber 26 cooperate to define biasing chamber 62. It should be appreciated that positioning sleeve 34 relative to sleeve 28 in the above described manner results in sleeve 34 being mechanically coupled to sleeve 28. In particular, sleeves 28 and 34 are mechanically coupled so that sleeve 28 is movable relative to sleeve 34 between a first position and a second position. For example, sleeve 34 can be slidably moved along a longitudinal axis 56 of bone distractor 12 in the direction indicated by arrow 58. Moving sleeve 34 in the aforementioned manner results in sleeve 34 being extended out of chamber 24 to a greater degree and thus results in sleeve 34 being located in an extended position as shown in FIG. 3. On the other hand, sleeve 34 can also be slidably moved along longitudinal axis 56 of bone distractor 12 in the direction indicated by arrow 60. Moving sleeve 34 in the direction indicated by arrow 60 results in sleeve 34 being retracted into chamber 24 to a greater degree and thus results in sleeve 34 being located in a retracted position as shown in FIG. 4. Accordingly, it should be appreciated that (i) when sleeve 34 is located in the first position relative to sleeve 28, sleeve 34 can be located in, for example, an extended position and (ii) when sleeve 34 is located in the second position relative to sleeve 28, sleeve 34 can be located, for example, in a retracted position.

As shown in FIG. 2, spontaneous biasing mechanism 18 is operatively coupled to bone distractor body 14 and bone distractor body 16. In particular, biasing mechanism 18 is positioned in biasing chamber 62 so that biasing mechanism 18 is mechanically coupled to sleeve 28 and sleeve 34. It should be appreciated that biasing mechanism 18 spontaneously urges bone distractor body 16 from the second position to the first position when distractor body 16 is located in the second position. What is meant herein by the phrase “biasing mechanism 18 spontaneously urges bone distractor body 16 from the second position to the first position” is that the urging communicated by biasing mechanism 18 to bone distractor body 16 occurs without the application of an external agency because of inherent properties of biasing mechanism 18. For example, biasing mechanism 18 continues to urge bone distractor body 16 in the above described manner in the absence of anyone or anything manipulating a component of bone distractor 12. This is in contrast to other devices utilized for bone distraction since these devices require the manipulation of a component of the device by an external agency, e.g. some of these devices require a surgeon to adjust a screw of the device to generate a bias.

Spontaneous biasing mechanism 18 can include a resilient body member 20 mechanically coupled to bone distractor body 14 and bone distractor body 16. What is meant herein by resilient body, is a body which, by virtue of its yield strength and elastic modulus, has the ability to recover its size and form following deformation. In particular, resilient body member 20 can be located within biasing chamber 62, i.e. chamber 24 and chamber 26, so that resilient body member 20 spontaneously urges bone distractor body 16 from the retracted position to the extended position when bone distractor body 16 is located in the retracted position. As shown in FIG. 2, spontaneous biasing mechanism 18 can include a spring 22 operatively coupled to bone distractor body 14 and bone distractor body 16. For example, spring 22 can be positioned within biasing chamber 62 such that ends 64 and 66 of spring 22 contact end wall 68 and end wall 70 of bone distractor body 14 and bone distractor body 16, respectively. It is also contemplated that biasing mechanism 18 can include other mechanisms for spontaneously urging bone distractor body 16 from the second position to the first position, these include, but are not limited to, a gas operatively coupled to bone distractor body 14 and bone distractor body 16 or a liquid a liquid operatively coupled to bone distractor body 14 and bone distractor body 16.

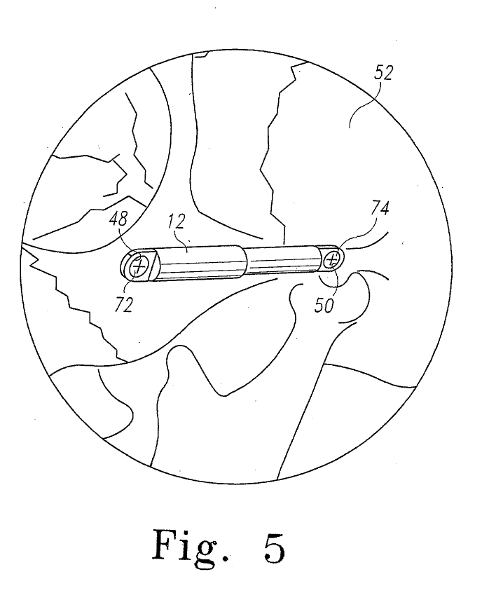

Referring now to FIG. 5, there is shown bone distractor 12 secured to a bone 52. In particular, bone distractor 12 is secured to bone 52 by (i) inserting a screw 72 through screw hole 48 and into bone 52 and (ii) inserting a screw 74 through screw hole 50 and into bone 52 so as to attach bone distractor 12 to bone 52.

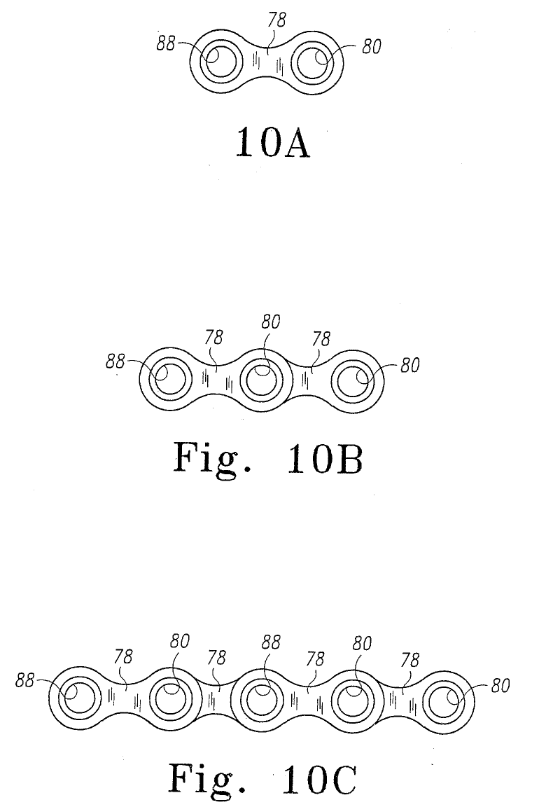

The following configurations utilize a number of attachment brackets 78 as shown in FIG. 10A. These attachment brackets 78 are positioned relative to one another in a cooperative manner, as shown in FIGS. 10B and 10C, such that one or more bone distractors 12 can be attached to a bone. However, it should be understood that a number of separate attachment brackets 78 can be utilized to attach a number of bone distractors 12 to a bone, or a single integrated attachment bracket configured to attach a number of bone distractors 12 to a bone can be utilized.

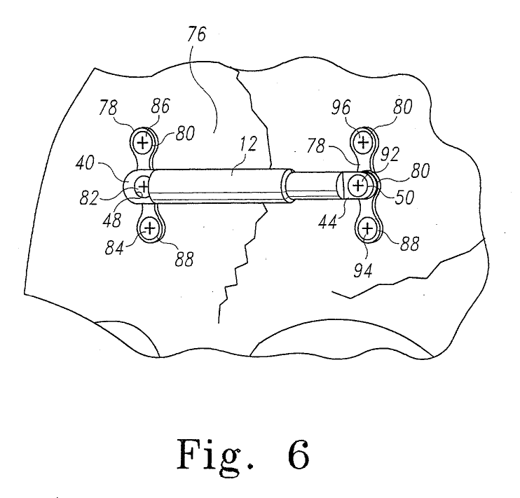

Referring now to FIG. 6, there is shown one configuration for securing bone distractor 12 to a bone 76 which utilizes attachment brackets 78. In particular attachment brackets 78 are positioned relative to one another as shown in FIG. 10B. These attachment brackets are then positioned relative to bone distractor 12 such that a central aperture 80 of attachment brackets 78 is aligned with screw hole 48 of bracket 40. Once aligned in the aforementioned manner a screw 82 is inserted through central aperture 80, screw hole 48, and into bone 76. Two additional screws 84 and 86 are also inserted through lateral apertures 88 and 80 of attachment brackets 78, respectively, so as to attach bone distractor 12 to bone 76. In a substantially identical manner, attachment brackets 78 are positioned relative to bone distractor 12 such that central aperture 80 of attachment brackets 78 is aligned with screw hole 50 of bracket 44. Once aligned in the aforementioned manner a screw 92 is inserted through central aperture 80, screw hole 50, and into bone 76. Two additional screws 94 and 96 are also inserted through lateral apertures 88 and 80 of attachment brackets 78, respectively, so as to attach bone distractor 12 to bone 76.

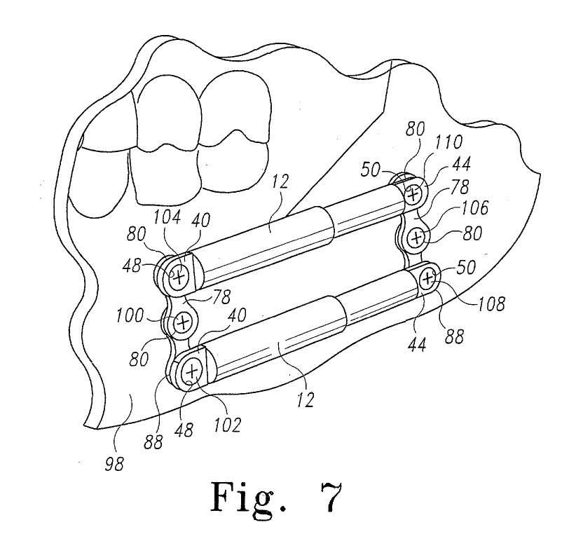

Referring now to FIG. 7, there is shown another configuration for securing multiple bone distractors 12 to a bone 98 utilizing attachment brackets 78. In particular, attachment brackets 78 are positioned relative to one another in the same manner as discussed above in reference to FIG. 10B. A first bone distractor 12 is positioned relative to attachment brackets 78 such that a lateral aperture 88 of attachment brackets 78 is aligned with screw hole 48 of bracket 40. In addition, attachment brackets 78 are also positioned relative to a second bone distractor 12 such that a lateral aperture 80 of attachment brackets 78 is aligned with screw hole 48 of bracket 40 of the second bone distractor 12. Once aligned in the aforementioned manner a screw 100 is inserted through central aperture 80 and into bone 98. Two additional screws 102 and 104 are also inserted (i) through lateral apertures 88 and 80 of attachment brackets 78, respectively, and (ii) through screw holes 48 of first and second bone distractors 12 so as to attach the first and second bone distractor 12 to bone 98. In a substantially identical manner, other attachment brackets 78 are positioned relative to first and second bone distractors 12 such that a lateral aperture 88 of attachment brackets 78 is aligned with screw hole 50 of bracket 44 of the first bone distractor. In addition, attachment brackets 78 are also positioned relative to the second bone distractor 12 such that a lateral aperture 80 of attachment brackets 78 is aligned with screw hole 50 of bracket 44 of the second bone distractor 12. Once aligned in the aforementioned manner a screw 106 is inserted through central aperture 80 and into bone 98. Two additional screws 108 and 110 are also inserted (i) through lateral apertures 88 and 80 of attachment brackets 78, respectively, and (ii) through screw holes 50 of first and second bone distractors 12 so as to further attach the first and second bone distractors 12 to bone 98.

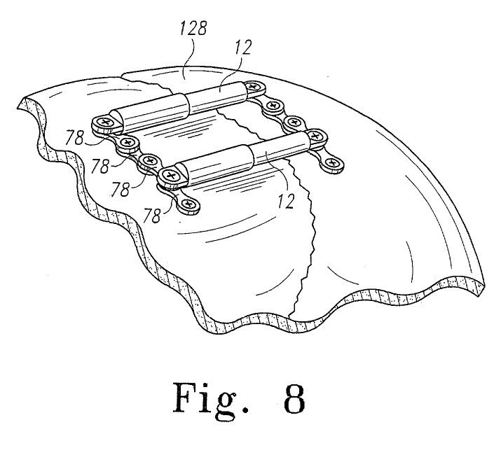

It should be appreciated that attachment configurations other than those described in reference to FIGS. 5, 6, and 7 are contemplated, including but not limited to the attachment configuration illustrated in FIG. 8 which utilizes a number of attachment brackets 78 (i.e. 4) to secure a pair of bone distractors 12 to a bone 128.

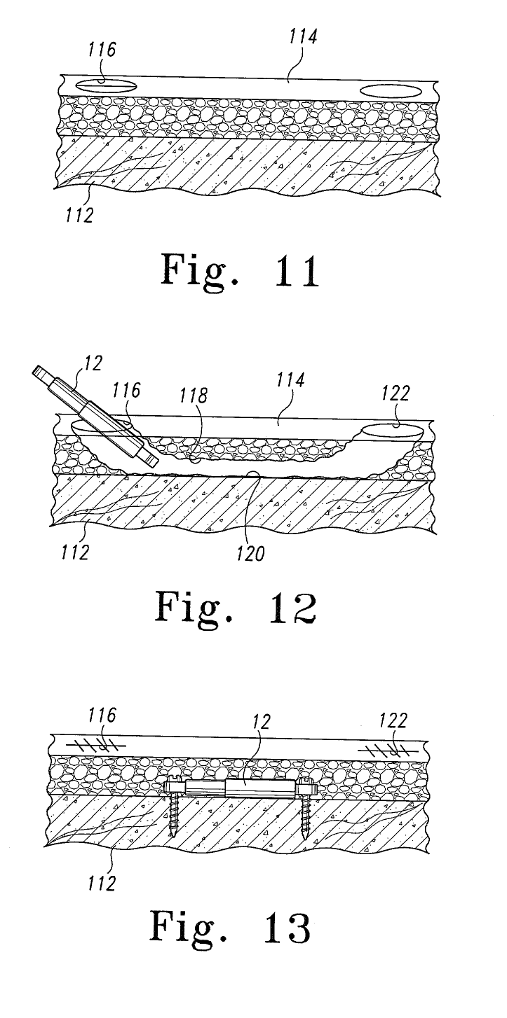

Now referring to FIGS. 11, 12, and 13, it should be appreciated that bone distractor 12 is secured to a bone 112 of an animal so that bone distractor 12 is completely underneath tissue 114 (e.g. skin) covering bone 112. For example, bone distractor 12 can be attached to a bone in the following manner. Initially, an incision 116 is created in skin 114, thereafter a tunnel 118 is created underneath skin 114 such that tunnel 118 runs along surface 120 of bone 112. As shown in FIGS. 12 and 13, bone distractor 12 is then inserted through incision 116 and into tunnel 118 such that bone distractor 12 is adjacent, and preferably in contact with, surface 120 of bone 112. Another incision 122 is created in skin 114 so that incision 122 provides an access to tunnel 118 and bone distractor 12. Once bone distractor 12 is positioned in the above described manner and incision 122 has been created, bone distractor 12 is secured to surface 120 of bone 112 utilizing, for example, one of the above described configurations described in reference to FIGS. 5-8. For example, bone distractor 12 can be secured to bone 112 by (i) inserting a screw through screw hole 48 of bracket 40 (see FIGS. 3 and 4) and into bone 112 and (ii) inserting a screw through screw hole 50 of bracket 44 (see FIGS. 3 and 4) and into bone 112 so as to attach bone distractor 12 to bone 112.

It should be appreciated that securing bone distractor 12 to bone 112 so that bone distractor 12 is positioned completely under skin 114, i.e. no portion of bone distractor 12 extends through skin 114 and is exposed to the outside environment, can alleviate the negative psychological impact experienced by patients who are subjected to a procedure that utilizes a device in which a portion of the device extends through the skin and is exposed to the outside environment. In addition, having bone distractor 12 positioned completely under skin 114 decreases the probability of infection at the surgical site, for example the pin tract infections experienced with devices that have pins extending through the skin of the patient.

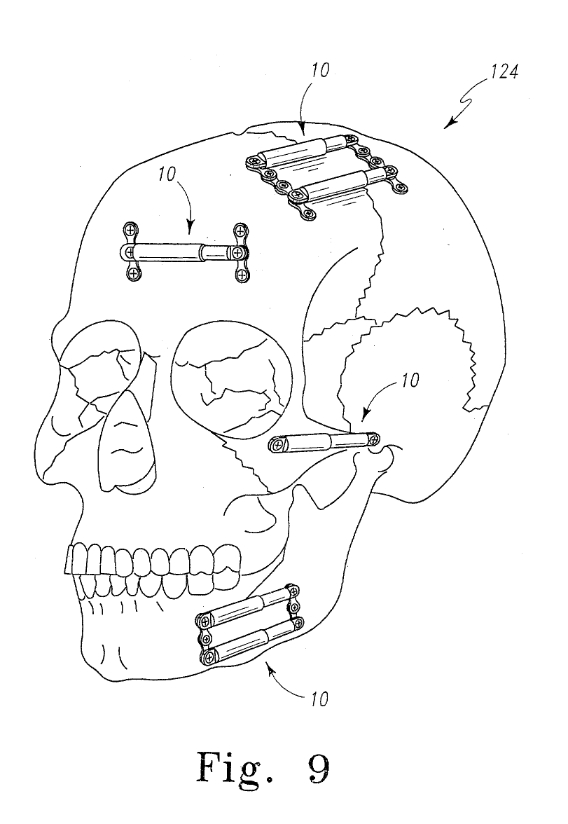

Still referring to FIG. 13, bone distractor 12 is secured to bone 112 in the above described manner so that bone distractor body 16 is located in a second position relative to bone distractor body 14. In particular, bone distractor 12 is secured to bone 112 such that bone distractor body 16 is located in a retracted position (see FIG. 4) relative to bone distractor body 14 when attached to surface 120 of bone 112. This can be accomplished by urging bone distractor body 16 into chamber 24 of bone distractor body 14 (e.g. pushing bone distractor body 16 into chamber 24) and then securing bone distractor 12 to bone 112 by inserting a screw through screw hole 50 of bracket 44 and into bone 112. Once bone distractor 12 is secured to bone 112 in this manner, bone distractor 12 communicates a force to bone 112 along longitudinal axis 56 in the direction indicated by arrows 58 and 60 (see FIG. 2). In particular, the aforementioned force is communicated to bone 112 as a result of spontaneous biasing mechanism 18 urging bone distractor body 16 from the retracted position to an extended position. It should be understood that this force results in bone 112 being expanded, e.g. lengthened, in the direction the force is applied. It should also be understood that the arrangement 10 for expanding a bone of an animal of the present invention can be utilized on various bones including, but not limited to, bones of a human skull 124 as shown in FIG. 9.

### EXAMPLE

Materials and Methods

Twelve, 30 day-old New Zealand white rabbits were used for this study. The animals were divided into two groups: 1) control group, (n=6), 2) experimental group (n=6). All the animals were anesthetized and the frontal and nasal bones were exposed by a midline incision. Two three-holed brackets were secured on the frontal nasal area 25 mm apart. In the experimental group a bone distractor 12 (40 mm long) was compressed to 25 mm (i.e. bone distractor body 16 was located in a retracted position) and secured to bone via the brackets. No bone distractor was secured to the bones of animals in the control group. The skin incisions were closed with interrupted 5-0 nylon sutures so that the bone distractor 12 was completely cover by the skin of the animal. All animals had a skull x-ray every week for four weeks. At the end of the fourth week, all surviving animals were sacrificed and the heads were defleshed for skull measurement and histological assessment.

Results

The results were evaluated clinically, radiographically, and histologically. Two of the animals in the experimental group developed incision infection and died. All surviving animals in the experimental group demonstrated progressive, marked midface protrusion and overbite. At the end of the fourth week, the distances between the two brackets varied from 37-39 mm in the experimental group and 30-32 mm in the control group. The average growth was 12±1 mm in the experimental group and 6±1 mm in the control group during the four-week distraction period. On histological observation, the distracted specimens contained more fibroblasts and osteoblasts with less organized fibrovascular matrix in comparison with controls.

While the invention has been illustrated and described in detail in the drawings and foregoing description, such an illustration and description is to be considered as exemplary and not restrictive in character, it being understood that only illustrative embodiments have been shown and described and that all changes and modifications that come within the spirit of the invention are desired to be protected.

There are a plurality of advantages of the present invention arising from the various features of the bone lengthening device described herein. It will be noted that alternative embodiments of the bone lengthening device of the present invention may not include all of the features described yet still benefit from at least some of the advantages of such features. Those of ordinary skill in the art may readily devise their own implementations of a bone lengthening device that incorporates one or more of the features of the present invention and fall within the spirit and scope of the present invention as defined by the appended claims.

What is claimed is:

1. An arrangement for expanding bone, comprising:
a bone distractor having (i) a first distractor body, (ii) a second distractor body mechanically coupled to said first distractor body so that said second distractor body is movable relative to said first distractor body between a first position and a second position, and (iii) a spontaneous biasing mechanism operatively coupled to said first distractor body and said second distractor body,
wherein said spontaneous biasing mechanism spontaneously urges said second distractor body from said second position to said first position when said second distractor body is located in said second position.

2. The arrangement of claim 1, wherein:
said spontaneous biasing mechanism includes a resilient body member mechanically coupled to said first distractor body and said second distractor body.

3. The arrangement of claim 2, wherein:
said spontaneous biasing mechanism includes a spring.

4. The arrangement of claim 2, wherein:
said first distractor body defines a first chamber,
said second distractor body defines a second chamber,
said second distractor body is slidably positioned within said first chamber such that said second distractor body is positionable between an extended position and a retracted position relative to said first distractor body,
said second distractor body is located in said extended position when located in said first position,
said second distractor body is located in said retracted position when located in said second position,
said resilient body member is located within said first chamber and said second chamber so that said resilient body member spontaneously urges said second distractor body from said retracted position to said extended position when said second distractor body is located in said retracted position.

5. The arrangement of claim 4, wherein:
said resilient body member includes a spring.

6. The arrangement of claim 4, wherein:
said first distractor body defines a first substantially cylindrical sleeve having a first end with a first opening defined therein, said first opening leading to said first chamber,
said second distractor body defines a second substantially cylindrical sleeve having a second end with a second opening defined therein,
said second opening leading to said second chamber, and
said second substantially cylindrical sleeve is positioned within said first chamber so that said second end of said second substantially cylindrical sleeve is positioned within said first chamber.

7. The arrangement of claim 6, further comprising:
a first bracket extending from a first attachment end of said first substantially cylindrical sleeve; and
a second bracket extending from a second attachment end of said second substantially cylindrical sleeve,
wherein (i) said first bracket has a first screw hole defined therein and (ii) said second bracket has a second screw hole defined therein.

8. A bone distractor, comprising:
a first bone distractor body having a first opening which leads to a first chamber defined in said first bone distractor body;
a second bone distractor body having a second opening which leads to a second chamber defined in said second bone distractor body, said second bone distractor body being slidably positioned within said first chamber so that (i) said second opening is located within said first chamber and (ii) said second bone distractor body is positionable relative to said first bone distractor body between an extended position and a retracted position; and
a spontaneous biasing mechanism operatively coupled to said first bone distractor body and said second bone distractor body,
wherein said spontaneous biasing mechanism spontaneously urges said second bone distractor body from said retracted position to said extended when said second bone distractor body is located in said retracted position.

9. The bone distractor of claim 8, wherein:
said spontaneous biasing mechanism includes a resilient body member mechanically coupled to said first bone distractor body and said second bone distractor body.

10. The bone distractor of claim 8, wherein:
said spontaneous biasing mechanism includes a spring operatively coupled to said first bone distractor body and said second bone distractor body.

11. The bone distractor of claim 8, wherein:
said spontaneous biasing mechanism includes a gas operatively coupled to said first bone distractor body and said second bone distractor body.

12. The bone distractor of claim 8, wherein:
said spontaneous biasing mechanism includes a liquid operatively coupled to said first bone distractor body and said second bone distractor body.

13. The bone distractor of claim 8, further comprising:
a first bracket extending from a first attachment end of said first bone distractor body; and
a second bracket extending from a second attachment end of said second bone distractor body,
wherein (i) said first bracket has a first screw hole defined therein and (ii) said second bracket has a second screw hole defined therein.

14. A method of expanding a bone of an animal, comprising:
inserting a bone distractor having (i) a first bone distractor body, (ii) a second bone distractor body mechanically coupled to said first bone distractor body so that said second bone distractor body is movable relative to said first bone distractor body between a first position and a second position, and (iii) a spontaneous biasing mechanism operatively coupled to said first bone distractor body and said second bone distractor body,
wherein said spontaneous biasing mechanism spontaneously urges said second bone distractor body from said second position to said first position when said second bone distractor body is located in said second position, underneath tissue covering said bone of said animal;
securing a first attachment end and a second attachment end of said bone distractor to said bone such that said second bone distractor body is located in said second position relative to said first bone distractor body; and
covering said bone distractor with said tissue so that said bone distractor is completely covered by said tissue.

15. A bone expanding device, comprising:
a first body having a first chamber defined therein;
a second body having a second chamber defined therein, said second body being slidably positioned within said first chamber so that said second body is positionable relative to said first body between an extended position and a retracted position; and
a resilient body member located within said first chamber and said second chamber so that said resilient body member spontaneously urges said second body from said retracted position to said extended position when said second body is located in said retracted position.

16. The bone expanding device of claim 15, wherein:
said resilient body member includes a spring.

17. The bone expanding device of claim 15, wherein:
said first body defines a first cylindrical sleeve, and
said second body defines a second cylindrical sleeve.

18. The bone expanding device of claim 17, further comprising:
a first bracket attached to said first cylindrical sleeve; and
a second bracket attached to said second cylindrical sleeve,
wherein (i) said first bracket has a first screw hole defined therein and (ii) said second bracket has a second screw hole defined therein.
